(19) United States
(12) Patent Application Publication
(10) Pub. No.: US 2025/0120632 A1
(43) Pub. Date: Apr. 17, 2025

(54) METHOD ADAPTED FOR DRIVER MONITORING SYSTEM AND DRIVER MONITORING SYSTEM

(71) Applicant: Harman International Industries, Incorporated, Stamford, CT (US)

(72) Inventors: Rengao ZHOU, Shanghai (CN); Hanyu SHANG, Shanghai (CN)

(21) Appl. No.: 18/834,223

(22) PCT Filed: Jan. 19, 2023
(86) PCT No.: PCT/CN2023/073150

§ 371 Date: Jul. 29, 2024

(30) Foreign Application Priority Data
Jan. 29, 2022 (WO) PCT/CN2022/074928

(51) Int. Cl. A61B 5/18; A61B 5/00; A61B 5/024; A61B 5/0255; G06T 7/00; G06T 7/90

(52) U.S. Cl. CPC A61B 5/18; A61B 5/02416; A61B 5/0255; A61B 5/6893; G06T 7/0012; G06T 7/90; A61B 2576/02; G06T 2207/10016; G06T 2207/10024; G06T 2207/30088; G06T 2207/30201; G06T 2207/30268

(57) ABSTRACT

The disclosure describes a method adapted for a driver monitoring system and the driver monitoring system. The method may comprise obtaining sequences of frames, wherein each sequence of frames consists of N arrays of color scalars corresponding to N face zones of a driver. For each array, the method may determine whether bad color scalars exist in the array; and perform interpolation adaptively to the array based on locations of bad color scalars in the array, in response to the determination of bad color scalars existing in the array.

### TECHNICAL FIELD

The present disclosure relates to a method adapted for a driver monitoring system (DMS) and the DMS using the method, and specifically relates to the method for accelerating the rPPG process in the DMS by using interpolation adaptively.

### BACKGROUND

The DMS is a popular and welcomed function in the field of ADAS (Advanced Driver Assistance System). It monitors and assesses the status of the driver while driving, and warns the driver if needed and eventually applies the brakes if necessary. The DMS is designed to detect two types of distractions: Visual Distraction (also known as ‘Eyes on Road’), and Cognitive Distraction (referred to as ‘Mind on Road’). Apart from the conventional method of analyzing eye gazes and eye blinks, cognitive load and stress could also be derived from the IBI, InterBeat-Interval (or simply speaking, the speed of heart beats). And the rPPG is a great approach of getting the IBI, since it doesn't need to contact the human skin.

The rPPG stands for remote-PhotoPlethysmoGraphy, or simply speaking, remote heart rate estimation. It is a simple optical technique used to detect volumetric changes in blood in peripheral circulation. The rPPG is measuring the variance of red, green, and blue light reflection changes from the skin, without contacting. This rPPG method measures the contrast between specular reflection and diffused reflection. The specular reflection is the pure light reflection from the skin, while the diffused reflection is the reflection that remains from the absorption and scattering in the skin tissue, which varies as blood volume changes.

In the actual environment, the data collected from people (such as, people's faces) for the rPPG process sometimes contains bad data caused by, for example, failed detection, or shades on the face. Thus, the existing rPPG method usually first determines whether the collected data contains bad data. If it determines that there is bad data, the existing rPPG process usually performs interpolation to make up for all the bad ones. Then, the existing rPPG process will perform subsequent method steps, such as performing FFT on the collected data which has been compensated using any existing interpolation algorithms.

However, applying the existing rPPG process under the scenarios of DMS in a vehicle may encounter some problems, and the most noticeable of them is the insufficiency of computing power of the DMS.

Therefore, it is necessary to provide an improved technology for not only saving computing power of the DMS to make real-time performance possible, but also increasing the accuracy of the rPPG algorithm.

### SUMMARY

According to one aspect of the disclosure, a method adapted for a driver monitoring system is provided. The method may comprise obtaining sequences of frames, wherein each sequence of frames consists of N arrays of color scalars corresponding to N face zones of a driver. For each array, the method may determine whether bad color scalars exist in the array; and perform interpolation adaptively to the array based on locations of bad color scalars in the array, in response to the determination of bad color scalars existing in the array.

According to another aspect of the present disclosure, a driver monitoring system is provided. The driver monitoring system may comprise a camera and a processor. The camera may capture face images of the driver in a vehicle. The processor may be coupled to the camera and obtain sequences of frames from the face images, wherein each sequence of frames consists of N arrays of color scalars corresponding to N face zones of the driver. The processor may perform the following for each array. The processor may determine whether bad color scalars exist in the array; and may perform interpolation adaptively to the array based on locations of bad color scalars in the array, in response to the determination of bad color scalars existing in the array.

According to another aspect of the present disclosure, a method for accelerating a remote-PhotoPlethysmoGraphy (rPPG) process in a driver monitoring system is provided. The method may comprise obtaining sequences of frames, wherein each sequence of frames consists of N arrays of color scalars corresponding to N face zones of a driver. For each array, the method may determine whether bad color scalars exist in the array; determine whether bad color scalars being at the end or the beginning of the array, in response to the determination of bad color scalars existing in the array; and discard the array in response to the bad color scalars being at the end or the beginning of the array, wherein the color scalars included in the discarded array are not used for a subsequent interpolation operation.

According to another aspect of the present disclosure, a driver monitoring system is provided. The driver monitoring system may comprise a camera and a processor coupled to the camera. The camera may be configured to capture face images of a driver in a vehicle. The processor may be configured to obtain sequences of frames from the face images, wherein each sequence of frames consists of N arrays of color scalars corresponding to N face zones of the driver. For each array, the processor may perform the following: determining whether bad color scalars exist in the array; determining whether bad color scalars being at the end or the beginning of the array in response to the determination of bad color scalars existing in the array; and discarding the array in response to the bad color scalars being at the end or the beginning of the array, wherein the color scalars included in the discarded array are not used for a subsequent interpolation operation.

In this way, responsive to the determinations for monitoring the driver warns the driver in response to the arrays of the driver face without the discarded arrays and/or applies a vehicle actuator, such as the vehicle brakes in response thereto.

According to yet another aspect of the present disclosure, a non-transitory computer-readable storage medium comprising computer-executable instructions is provided which, when executed by a computer, causes the computer to perform the method disclosed herein.

### BRIEF DESCRIPTION OF THE DRAWINGS

FIG. 1 illustrates an example of the definition of landmarks and the generated face zones.

FIG. 2 illustrates an example of the expected final calculated color scalars.

FIG. 3 illustrates examples of sequences of frames and the corresponding expected plotted diagrams which should correspond to the actual heart beats.

FIG. 4 illustrates an example of erroneous estimation resulting from not discarding the array with bad color scalars located at its beginning.

FIG. 5 illustrates a flowchart of the method performed for each array according to one or more embodiments of the present disclosure.

FIG. 6 illustrates a flowchart of the method of performing interpolation adaptively according to one or more embodiments of the present disclosure.

FIG. 7 illustrates a flowchart of the method of utilizing the timespan of heart beat when discarding the array in response to bad color scalars existing at the end of the array, according to one or more embodiments of the present disclosure.

FIG. 8 illustrates an exemplary case in which the time period of bad color scalars is larger than the timespan of heart beat.

FIG. 9 illustrates another flowchart of the method of performing interpolation adaptively according to one or more embodiments of the present disclosure.

FIG. 10 illustrates an example DMS.

To facilitate understanding, identical reference numerals have been used, where possible, to designate identical elements that are common to the figures. It is contemplated that elements disclosed in one embodiment may be beneficially utilized on other embodiments without specific recitation. The drawings referred to here should not be understood as being drawn to scale unless specifically noted. Also, the drawings are often simplified and details or components omitted for clarity of presentation and explanation. The drawings and discussion serve to explain principles discussed below, where like designations denote like elements.

### DETAILED DESCRIPTION OF THE PREFERRED EMBODIMENTS

Examples will be provided below for illustration. The descriptions of the various examples will be presented for purposes of illustration, but are not intended to be exhaustive or limited to the embodiments disclosed. Many modifications and variations will be apparent to those of ordinary skill in the art without departing from the scope and spirit of the described embodiments.

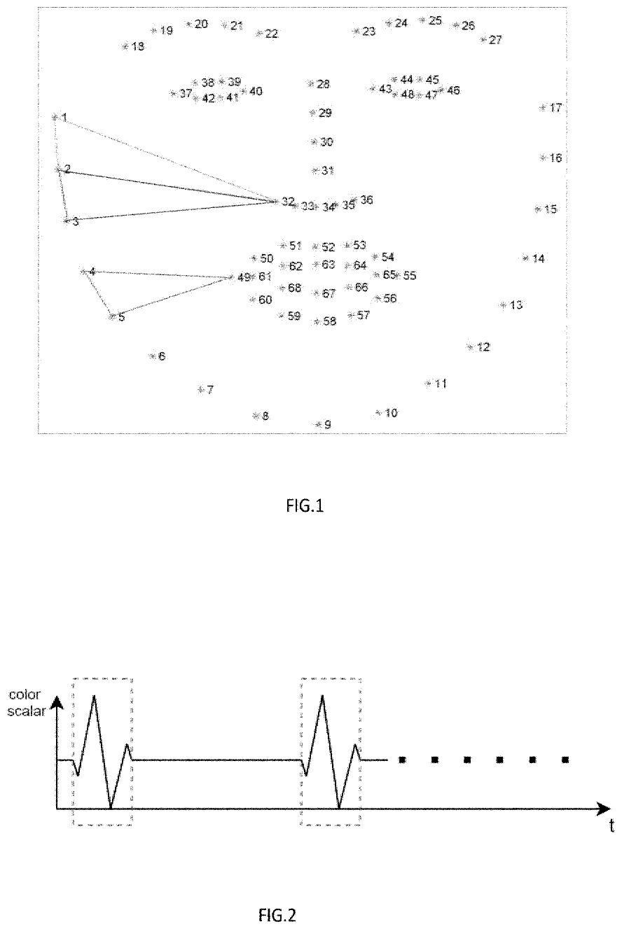

In the present disclosure, a novel method and system is provided to accelerate the rPPG process/algorithm to address the issues faced when porting it into a DMS on a vehicle. This is achieved by using interpolation adaptively based on the determination of locations of bad color scalars in the arrays corresponding to face zones of the driver, instead of doing interpolation to make up for all the bad color scalars. The innovation proposed in this disclosure may improve the computing speed, save computing power, thereby making real-time performance possible. Furthermore, the approach proposed in the present disclosure may exclude erroneous estimations of rPPG signals due to bad color scalars. As a result, the speed as well as the accuracy of the rPPG algorithm are improved. The novel approach will be explained in detail referring to FIGS. 1-9 as follows.

Under the scenarios of DMS, the rPPG process takes the color scalars of different predefined face zones as input, wherein the color scalars are values related to the light reflection changes from the skin. These predefined face zones are generated by linking the detected face landmarks. FIG. 1 illustrates an example of the definition of landmarks and the generated face zones. As shown in the example of FIG. 1, for a face, 68 face landmarks may be detected, and accordingly the face may be partitioned into 51 predefined face zones. In FIG. 1, only several predefined face zones are marked with triangles for clarity of presentation.

For example, a sequence of frames will be considered or processed together, for color scalars of every 51 predefined face zones. Thus, there will be a total of 51 arrays of color scalars at a time. For those face zones in which the color scalars are bad (usually because of failed detection, or because of leafy shades on driver's face, and so on), an interpolation will be used to make up for the bad ones. After that, FFT (Fast Fourier Transfer) will be applied over this sequence of color scalars. Based on the result of FFT, the SNR (Signal-Noise Ratio) values may be calculated. Each FFT may also be fed into a Neural Network to derive the signal quality. And all 51 color scalars will be added up to calculate the final values indicating the actual heart beats.

Ideally, a total of 51 FFTs will be fed into the Neural Network, meaning the Neural Network will be computed 51 times for every frame sequence. And upon the arrival of new frame, the data of new frame will be appended to the end of sequence while the beginning of sequence will be deleted to keep the fixed length of the sequence. As a result, the Neural Network will take up a huge percentage of the total computing force. And this above process will be triggered every time a new sequence of frames arrive. Considering the limited computing force in the vehicle, real-time performance may not be guaranteed.

In order to solve this problem, this disclosure proposes a new approach. Since the rPPG measures the contrast between the specular reflection and diffused reflection, and the diffused reflection is the reflection that remains from the absorption and scattering in the skin tissue, which varies as blood volume changes, the calculated final color scalars will also varies as the blood volume changes. Simply speaking, the calculated final color scalars correspond to the heart beats, acting somewhat like pulse waves. An example of the expected final calculated color scalars is shown in the FIG. 2. In FIG. 2, areas marked by dashed rectangles are ‘pulse waves’ resulted from heart beats. And it can be noticed that, apart from the parts of pulse waves resulted from the heart beats, other parts are roughly the same. Those parts are useless for interpolation. Thus, other than directly applying interpolation to the missing parts of color scalars, this innovation proposes new algorithms.

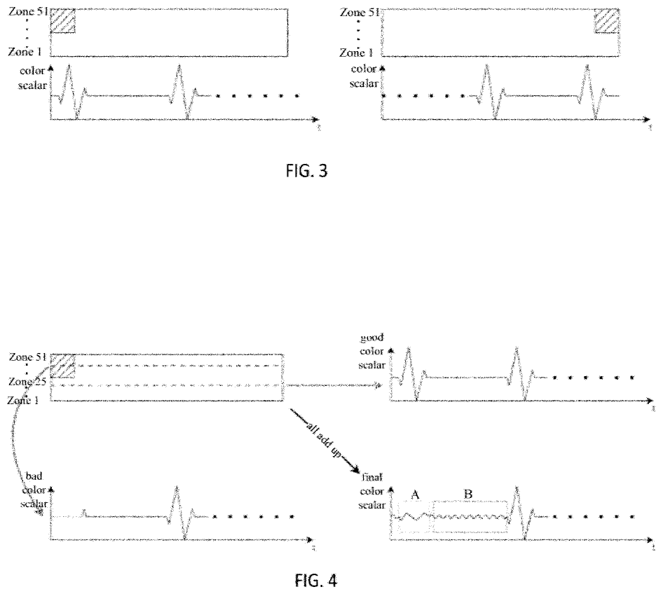

FIG. 3 illustrates examples of sequences of frames and the corresponding expected plotted diagrams of color scalars which should correspond to the actual heart beats. For example, among a sequence of frames, there are 51 arrays of color scalars for 51 predefined face zones. As shown in FIG. 3, the top-left image shows the case where there are bad color scalars at the beginning of some arrays, marked by shadowed rectangle, wherein the horizontal axis direction represents the length of the frame sequence, and the vertical axis direction represents 51 face zones and also represents 51 arrays (such as Array 1 to Array 51) corresponding to the 51 face zones (such as Zone 1 to Zone 51). Understandably, each array may have multiple color scalars for the duration of the entire frame sequence (i.e., the length of the frame sequence). The bottom-left image shows the expected plotted diagram of color scalars corresponding to the sequence of frames shown in the top-left image. Likewise, the top-right image shows the cases of bad color scalars at the end of some arrays, and the bottom-right image shows the expected plotted diagram of color scalars corresponding to the sequence of frames shown in the top-right image.

In the proposed approach, if there are bad color scalars at the beginning of specific array (even if these parts are also corresponding to the parts containing pulse waves in other arrays), then this specific whole array will not go into the following steps (such as interpolation, FFT, SNR, Neural Network, and so on), since lacking the values ahead of the beginning of array to do the interpolation. If there are bad color scalars at the end of specific array (even if these parts are also corresponding to the parts containing pulse waves in other arrays), then this specific whole array will neither go into the following steps, since lacking the values after the end of array to do the interpolation. In addition, as shown in the FIG. 3, if there are bad color scalars at the beginning or at the end of the array, although the other parts of the array might contain proper values of color scalars, the whole array will be discarded and will not go into the following steps (such as interpolation, FFT, SNR, Neural Network, and so on), because the interpolation in this situation will not be able to make up for the missing values.

FIG. 4 illustrates an example of erroneous estimation resulting from not discarding the array with bad color scalars located at its beginning. The top-left image in FIG. 4 is an example of the 51 arrays of color scalars corresponding to 51 face zones, wherein there are some bad color scalars containing heart beat (i.e., containing heart beat information) at the beginning of some arrays. As an example, all the color scalars of Array 1-Array 25 corresponding to Zone 1-Zone 25 are good color scalars, while the color scalars of Array 26-Array 51 corresponding to Zone 26-Zone 51 include some bad color scalars (marked by shadowed rectangle) and some good color scalars. The top-right image in FIG. 4 is the plotted diagram corresponding to all good color scalars of Array 1-Array 25. The bottom-left image is the plotted diagram corresponding to Array 26-Array 51 which contain some bad color scalars. The bottom-right image is the final color scalar, which is calculated from all 51 arrays.

As can be seen from FIG. 4, the contrast between part A and part B of the final results are not noticeable enough, as the stable parts of scalars may also contain noise, shown as waves in part B. As a result, part A may not be considered to be heart beat, but it should be. Thus, adding an array which contains parts of bad values and parts of proper values to the final results, will make the final results unreliable, and may lead to bad results. Therefore, it's better to discard such whole array, and preclude it from going into the following steps (such as interpolation, FFT, SNR, Neural Network, and so on). Discarding the whole array like this would not only save computing power, but also exclude cases of wrong results.

FIG. 5 illustrates a flowchart of the method performed for each array according to one or more embodiments of the present disclosure. At S502, an estimation may be first performed to determining whether bad color scalars exist in the array. As mentioned above, the color scalars are values related to the light reflection changes from the skin. It may be understandable that the appearance of bad color scalars means the appearance of bad values in a sequence of frames including collected color scalars. As those skilled in the art know, the appearance of bad values is caused by the failed face detection or leafy shadows on face.

Generally, there are two cases of failed face detection. In one case, the face detection function for the whole face does not detect a human face. In the other case, although the face detection detects the face, there is no value for the hidden part caused by the face turning sideways, which is also classified as failed face detection. That is to say, in the situation of failed face detection, if the face is not detected at the time corresponding to a certain frame or frames, there is no value in the corresponding frame or frames. If there is no value, it can be considered that there are bad values in the frame sequence. In other words, it can be determined that there are bad color scalars in the array.

As for the situation of leafy shadows on face, it will make a part of the face have shadows, which will cause the value of the shadow part to be quite different from that of other normal parts. Because the shadow is black, that is, the value of color scalar is biased towards 0.

In view of the above two situations of bad value generation, the method often adopted by the skilled person in the field is to determine whether there is bad color scalar by determining whether there is any color scalar with no value or very small value in the array (i.e., in the sequence of frames). The very small value, for example, can be compared with the average value of all the values of the color scalar in the array. Those skilled in the art can preset a proportional threshold according to their practice experience. For example, when the ratio of the value of the color scalar to the average value is less than the proportional threshold (e.g., 1/2), the value of this color scalar is considered as a bad value. It can be understood by those skilled in the art that since the bad value in a frame sequence can be found, the location where the bad value appears in the frame sequence can of course be obtained. In other words, the location where the bad color scalar appears in the array can be obtained.

At S504, an interpolation to the array may be performed adaptively in response to the determination of bad color scalars existing in the array. According to one or more embodiment, the interpolation may be performed adaptively based on locations of bad color scalars in the array.

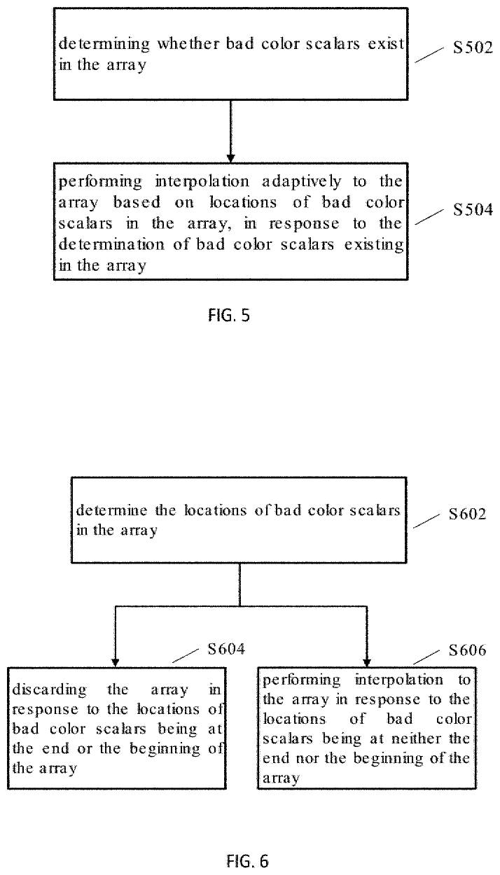

FIG. 6 illustrates a flowchart of the method of performing interpolation adaptively according to one or more embodiments of the present disclosure. At S602, an estimation may be performed to determine the locations of bad color scalars in the array. As discussed above with reference to FIG. 5, it can be understood by those skilled in the art that since the bad color scalar in the array can be found, the location where the bad color scalar appears in the array can of course be obtained.

If the locations of bad color scalars are at the end or the beginning of the array (i.e., there are bad color scalars containing heart beat at the end of the array, or there are bad color scalars containing heart beat at the beginning of the array), then the array will be discarded, at S604. If the locations of bad color scalars are at neither the end nor the beginning of the array, then the interpolation will be performed to make up for bad color scalars, at S606. As described above with reference to FIG. 5, the causes of bad value include two situations of failed face detection and shadows on face. Both of these two situations will not end immediately, but will last for some time. That is to say, the appearance of bad value will not be isolated, but will continue for a period of time. This is because whether the face deflects sideways or the environment outside the car produces shadows, it is a continuous condition instead of a discrete condition. Therefore, there is usually no case where a frame of good value and a frame of bad value jump around, but there will be cases where either good values or bad values appear continuously for a period of time. Therefore, a simple way to determine whether the bad values appears at the end or the beginning of a frame sequence (i.e., whether the locations of bad color scalars are at the end or the beginning of the array) is to determine whether the value of color scalar at the last frame or the first frame of the frame sequence is bad value.

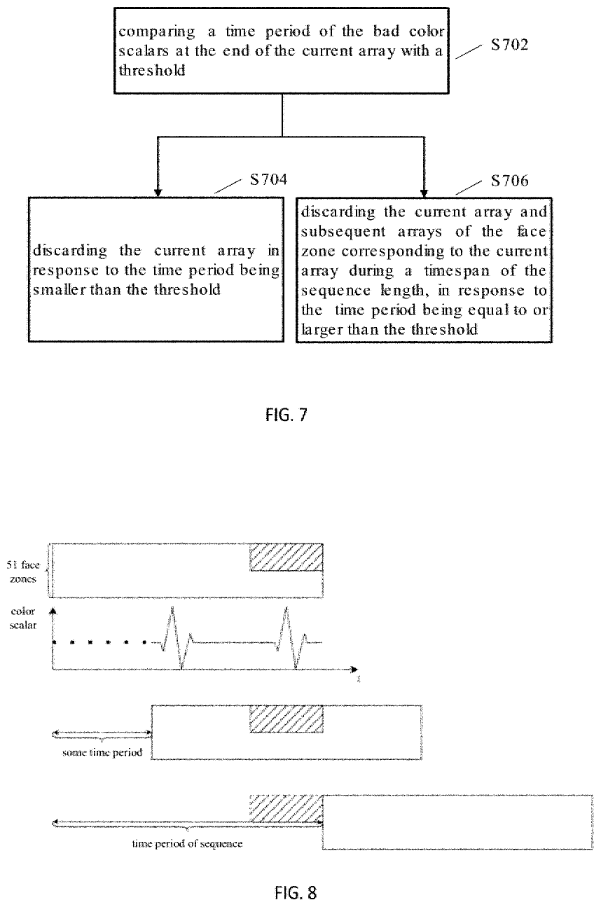

Moreover, the disclosure proposes a preferable method to further accelerating the rPPG computing and improving the accurency of the rPPG computation. FIG. 7 illustrates a flowchart of this method of utilizing the timespan of heart beat when disregarding the array in response to bad color scalars existing at the end of the array, according to one or more embodiments of the present disclosure. At S702, a time period of the bad color scalars at the end of the current array is compared with a threshold. If the time period of the bad color scalars is smaller than the threshold, the current array is discarded, at S704. If the time period is equal to or larger than the threshold, then at S706, during a timespan of the sequence length, the current array and subsequent arrays of the face zone corresponding to the current array are discarded. In other word, if the time period of bad color scalars at the end of array is larger than the threshold, then the arrays for this corresponding face zone, in all the following sequences would be neglected, until a length of sequence has been passed away.

According to one or more embodiments, the threshold may adaptively alter according to the timespan of a heart beat (i.e., the whole period of the heart beat), for example, the width of the dashed rectangles in FIG. 2 corresponds to the time span of the heartbeat. For example, if the timespan of heart beat is smaller, the threshold may be changed to be smaller. If the timespan of heart beat is larger, the threshold may be changed to be larger. Thus, keeping record of the previous timespan of heart beat, and updating it upon new arrival of heart beats, would help to adaptively alter the threshold and accordingly alter the process.

FIG. 8 illustrates an exemplary case in which the time period of bad color scalars is longer than the timespan of heart beat. As shown in FIG. 8, the bad color scalars contains a whole time period of heart beat. If the bad color scalars are longer than the time period of heart beat, then it means a whole process of heart beat is missing, and the interpolation cannot make up for it. And these bad color scalars would persist in the current array and subsequent arrays of the face zone corresponding to the current array, until totally passed away out of the sequence. That means this interpolation failing would persist with new frames coming into the sequence, until these bad color scalars have been passed away out of the sequence.

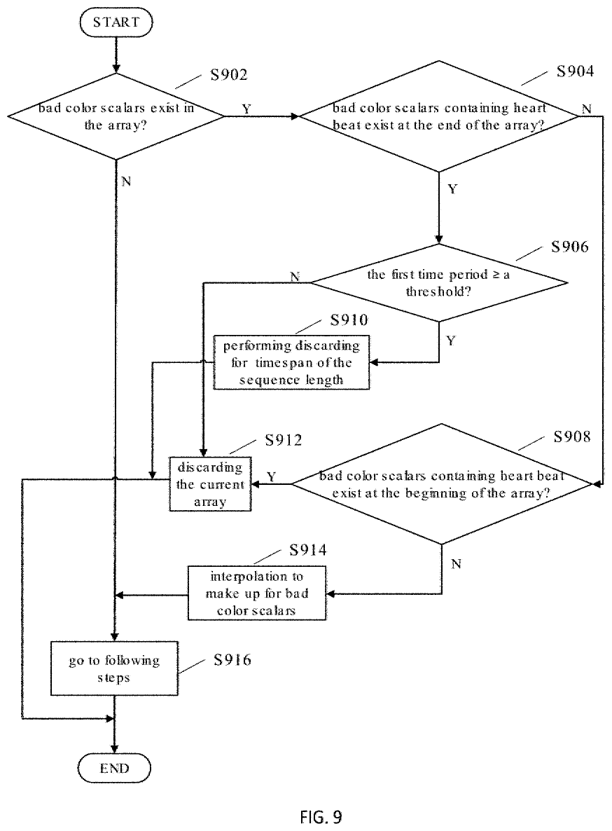

FIG. 9 illustrates another flowchart of the method of performing interpolation adaptively according to one or more embodiments of the present disclosure. As shown in FIG. 9, at S902, an estimation may be performed to determine if bad color scalars exist in the array. If it determines that bad color scalars exist in the array, then the method goes to S904. If it determines at S902, that no bad color scalars exist in the array, then the method goes to S916 for going to the following steps. At S916, the following steps may comprise, for example, at least one of the processing steps for FFT, SNR and Neural Network.

At S904, an estimation may be performed to determine if bad color scalars containing heart beat are located at the end of the array. If it determines at S904 that bad color scalars containing heart beat are located at the end of the array, then the method goes to S906.

At S906, a comparison may be performed to determine whether a first time period is equal to or greater than a threshold, wherein the first time period is the time period of the bad color scalars at the end of the array. The threshold may vary adaptively based on a timespan of heart beat. If it determines at S906 that the time period of the bad color scalars at the end of the array is equal to or greater than the threshold, then the method goes to S910. At S910, the discarding process is performed for the timespan of the sequence length. That means, during the timespan of the sequence length, the current array and subsequent arrays of the face zone corresponding to the current array are discarded. Then, the method goes to the end.

If it determines at S906 that the time period of the bad color scalars at the end of the array is smaller than the threshold, then the method goes to S912. At S912, the current array is discarded. Then, the method goes to the end.

If it determines at S904 that bad color scalars containing heart beat are not located at the end of the array, then the method goes to S908. At S908, an estimation may be performed to determine if bad color scalars corresponding to heart beat are located at the beginning of the array. If it is determined at S908 that bad color scalars corresponding to heart beat are located at the beginning of the array, then the method goes to S912. At S912, the current array is discarded.

If it is determined at S908 that bad color scalars corresponding to heart beat are not located at the beginning of the array, then the method goes to S914. At S914, an interpolation is performed to make up for bad color scalars. Then, the method goes to S916 for going to the following steps. It can be understood that the specific determination methods adopted at S902, S904 and S908 are the same as those described above with reference to FIGS. 5-6, and will not be repeated here.

The method described in this disclosure may be adapted for the DMS. For example, the vehicle DMS 1010 of FIG. 10 may comprise a camera 1012 and a processor and memory 1014 coupled to the camera. The camera may be coupled in a vehicle cabin facing one or more of the driver and passengers and may capture face images of the driver in a vehicle. The processor may be coupled with the memory, and the memory may include instructions to perform all the methods described above. For example, the processor may process the captured images and obtain sequences of frames, wherein each sequence of frames consists of N arrays of color scalars corresponding to N face zones of the driver. The processor may perform the following steps for each array. The processor may determine whether bad color scalars exist in the array; and may perform interpolation adaptively to the array in response to the determination of bad color scalars existing in the array; wherein the performing interpolation adaptively to the array may be based on locations of bad color scalars in the array. The processor may further be coupled to actuators to generate a warning and/or actuate a vehicle actuator, such as a vehicle brake, in response to the interpolated arrays without the discarded arrays. For example, the processor may include instructions to monitor the face of the driver based on the frames but without the discarded array.

By discarding an array, and the not using the discarded array in subsequent interpolation, the processor can more efficiently monitor the face of the driver with high accuracy since less array data (particularly that which may cause erroneous monitoring) is utilized. This is particularly applicable where there are issues as to the insufficiency of computing power of the DMS, and thus the approach described herein solves a particularly difficult technical problem.

The processor may be any technically feasible hardware unit configured to process data and execute software applications, including without limitation, a central processing unit (CPU), a microcontroller unit (MCU), an application specific integrated circuit (ASIC), a digital signal processor (DSP) chip and so forth.

1. In some embodiments, a method adapted for a driver monitoring system comprising: obtaining sequences of frames, wherein each sequence of frames consists of N arrays of color scalars corresponding to N face zones of a driver; and for each array, performing the following: determining whether bad color scalars exist in the array; and performing interpolation adaptively to the array based on locations of bad color scalars in the array, in response to the determination of bad color scalars existing in the array.

2. The method according to clause 1, wherein the performing interpolation adaptively to the array comprises: determining the locations of bad color scalars in the array; and discarding the array in response to the locations of bad color scalars being at the end or the beginning of the array; or performing interpolation to the array in response to the locations of bad color scalars being at neither the end nor the beginning of the array.

3. The method according to any one of clauses 1-2, wherein the discarding the array in response to the locations of bad color scalars being at the end or the beginning of the array comprises: comparing a first time period of the bad color scalars at the end of the array with a threshold if the bad color scalars exist at the end of the array; and discarding the current array in response to the first time period being smaller than the threshold; or discarding the current array and subsequent arrays of the face zone corresponding to the current array during a second time period, in response to the first time period being equal to or larger than the threshold.

4. The method according to any one of clauses 1-3, wherein the second time period is equal to the length of the sequence.

5. The method according to any one of clauses 1-4, wherein the threshold alters adaptively based on a timespan of heart beat.

6. The method according to any one of clauses 1-5, further comprises: keeping a record of the previous timespan of heart beat; and updating the record upon a new arrive of heart beat.

7. The method according to any one of clauses 1-6, further comprises: capturing face images of the driver by a camera of the driver monitoring system in a vehicle; and obtaining the sequences of frames from the face images.

8. The method according to any one of clauses 1-7, wherein the method is utilized to accelerate a remote-PhotoPlethysmoGraphy (rPPG) process in the driver monitoring system.

9. A driver monitoring system comprising: a camera configured to capture face images of a driver in a vehicle; and a processor coupled to the camera and configured to: obtain sequences of frames from the face images, wherein each sequence of frames consists of N arrays of color scalars corresponding to N face zones of the driver; and for each array, perform the following: determining whether bad color scalars exist in the array; and performing interpolation adaptively to the array based on locations of bad color scalars in the array, in response to the determination of bad color scalars existing in the array.

10. The driver monitoring system according to clause 9, wherein the processor is configured to determine the locations of bad color scalars in the array; and discard the array in response to the locations of bad color scalars being at the end or the beginning of the array; or perform interpolation to the array in response to the locations of bad color scalars being at neither the end nor the beginning of the array.

11. The driver monitoring system according to any one of clauses 9-10, wherein the processor is configured to: compare a first time period of the bad color scalars at the end of the array with a threshold if the bad color scalars exist at the end of the array; and discard the current array in response to the first time period being smaller than the threshold; or discard the current array and subsequent arrays of the face zone corresponding to the current array during a second time period, in response to the first time period being equal to or larger than the threshold.

12. The driver monitoring system according to any one of clauses 9-11, wherein the second time period is equal to the length of the sequence.

13. The driver monitoring system according to any one of clauses 9-12, wherein the threshold alters adaptively based on a timespan of heart beat.

14. The driver monitoring system according to any one of clauses 9-13, wherein the processor is configured to: keep a record of the previous timespan of heart beat; and update the record upon a new arrive of heart beat.

15. A computer-readable storage medium comprising computer-executable instructions which, when executed by a computer, causes the computer to perform the method according to any one of claims 1-8.

The descriptions of the various embodiments have been presented for purposes of illustration, but are not intended to be exhaustive or limited to the embodiments disclosed. The terminology used herein was chosen to best explain the principles of the embodiments, the practical application or technical improvement over technologies found in the marketplace, or to enable others of ordinary skill in the art to understand the embodiments disclosed herein.

In the preceding, reference is made to embodiments presented in this disclosure. However, the scope of the present disclosure is not limited to specific described embodiments. Instead, any combination of the preceding features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the preceding aspects, features, embodiments and advantages are merely illustrative and are not considered elements or limitations of the appended claims except where explicitly recited in a claim(s).

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module”, “unit” or “system.”

The present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective calculating/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the drawings illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the foregoing is directed to embodiments of the present disclosure, other and further embodiments of the disclosure may be devised without departing from the basic scope thereof, and the scope thereof is determined by the claims that follow.

1. A driver monitoring system, comprising:
a camera positioned in a vehicle to capture face images of a driver in the vehicle; and
a processor coupled to the camera and configured to:
obtain sequences of frames from the face images, wherein each sequence of frames includes N arrays of color scalars corresponding to N face zones of the driver; and
for each array, perform the following:
determining whether bad color scalars exist in the array;
determining whether bad color scalars being at the end or the beginning of the array, in response to the determination of bad color scalars existing in the array; and
discarding the array in response to the bad color scalars being at the end or the beginning of the array, wherein the color scalars included in the discarded array are not used for a subsequent interpolation operation.

2. The driver monitoring system according to claim 1, wherein the processor is configured to:
compare a first time period of the bad color scalars at the end of the array with a threshold if the bad color scalars exist at the end of the array; and
discard the current array in response to the first time period being smaller than the threshold; or
discard the current array and subsequent arrays of the face zone corresponding to the current array during a second time period, in response to the first time period being equal to or larger than the threshold.

3. The driver monitoring system according to claim 2, wherein the second time period is equal to the length of the sequence.

4. The driver monitoring system according to claim 2, wherein the threshold alters adaptively based on a timespan of heart beat.

5. The driver monitoring system according to 4, wherein the processor is configured to:
keep a record of the previous timespan of heart beat; and
update the record upon a new arrive of heart beat.

6. A method for accelerating a remote-PhotoPlethysmoGraphy (rPPG) process in a driver monitoring system, comprising:
obtaining sequences of frames, wherein each sequence of frames consists of N arrays of color scalars corresponding to N face zones of a driver; and
for each array, performing the following:
determining whether bad color scalars exist in the array;
determining whether bad color scalars being at the end or the beginning of the array, in response to the determination of bad color scalars existing in the array;
discarding the array in response to the bad color scalars being at the end or the beginning of the array, wherein the color scalars included in the discarded array are not used for a subsequent interpolation operation; monitoring the face of the driver based on frames but without the discarded array.

7. The method according to claim 7, wherein the discarding the array in response to the bad color scalars being at the end or the beginning of the array comprises:
comparing a first time period of the bad color scalars at the end of the array with a threshold if the bad color scalars exist at the end of the array; and
discarding the current array in response to the first time period being smaller than the threshold; or
discarding the current array and subsequent arrays of the face zone corresponding to the current array during a second time period, in response to the first time period being equal to or larger than the threshold.

8. The method according to claim 8, wherein the second time period is equal to the length of the sequence.

9. The method according to claim 8, wherein the threshold alters adaptively based on a timespan of heart beat.

10. The method according to claim 7, further comprises:
keeping a record of the previous timespan of heart beat; and
updating the record upon a new arrive of heart beat.

11. The method according to claim 7, further comprises:
capturing face images of the driver by a camera of the driver monitoring system in a vehicle; and
obtaining the sequences of frames from the face images.

7-12. (canceled)
